(19) United States
(12) Patent Application Publication
(10) Pub. No.: US 2009/0183738 A1
(43) Pub. Date: Jul. 23, 2009

(54) Device for Complex Interval Normobaric Hypoxic Hyperoxic Training of a Human

(75) Inventors: Alexander I. Kostin, Moscow (RU); Oleg S. Glazachev, Moscow (RU); Alexey V. Platonenko, Moscow (RU); Galina K. Spirina, Moscow (RU)

Correspondence Address: HOUSTON ELISEEVA, 4 MILITIA DRIVE, SUITE 4, LEXINGTON, MA, 02421, US

(73) Assignee: VNIIMI GROUP OF COMPANIES, Moscow (RU)

(21) Appl. No.: 12/015,840

(22) Filed: Jan. 17, 2008

(51) Int. Cl. A61M 16/00

(52) U.S. Cl. 128/204.18

(57) ABSTRACT

This invention is directed to medical equipment and can be used for elevation of resistance of a human body against various pathologies including bronchial asthma, hyperplastic anemia and iron deficiency anemia, neurocirculatory dystonia, hypertension, obesity. This instrument can also be useful for treatment and prevention of respiratory and metabolic dysfunctions, for enhancement of non-specific compensatory capabilities of a human body and its aerobic output, as well as for improving physical fitness and regenerative ability of athletes, and reducing negative side effects of ionizing radiation.

### BACKGROUND OF THE INVENTION

This invention is related to the field of medical equipment and can be used for elevation of resistance of a human body against various pathologies including bronchial asthma, hyperplastic anemia and iron deficiency anemia, neurocirculatory dystonia, hypertension, obesity. The invention can also be useful for treatment and prevention of respiratory and metabolic dysfunctions, for enhancement of non-specific compensatory capabilities of a human body and its aerobic output, as well as for improving physical fitness and regenerative ability of athletes, and for reducing negative side effects of ionizing radiation.

Methods and instruments for normobaric oxygen therapy and training, during which an oxygen-enriched gas mixture is administered to the patient are widely used in the medical field.

It was also observed that various methods of hypoxic training (respiratory training with periodic and interval exposure to hypoxic gas mixtures) are used for improvement of physiological mechanisms of oxygen transport and utilization.

Existing devices, however, do not allow for altering the gas mixture composition with respect to oxygen concentration depending on the patient's condition or by an operator.

The closest analog to the proposed device is a device for integrated oxygen- and reduced-oxygen therapy.

The device includes a compressor used as a source of compressed air connected to a gas separation module, and valve, which in turn is connected to the usage gauge. The gas separation module is equipped with a packet of gas separation membranes, which allows for output of both hypo- and hyperoxic mixtures. The two output channels are connected to output jets, one directly, another though the valve and a usage gauge. A respiration unit of the device comprises a flow switch, wherein input channels are connected to output jets of the device, and the flow switch is connected to a control unit. In addition to the compressor used as a source of compressed air, the device may contain a vacuum pump/compressor for redirection of the output flow into the compressor input.

There are, however, limitations associated with described design: utilization of an additional vacuum pump/compressor leads to increased mass and overall dimensions of the apparatus, as well as high maintenance and operation requirements. In addition, the hypoxic/hyperoxic flow switch is joined with the respiration unit, which makes the device inconvenient for patient's use. The flow switch is not equipped with vents for hypo- or hyperoxic flow channels, which may cause changes in the initial mode of operation of the gas separation membranes. The programs entered into the control unit governing the hypo- and hyperoxic gas mixture delivery provide periodic alternation between the two types of gas mixtures based on preset parameters; the parameters do not adjust automatically to the patient's current condition while the patient is being exposed to hypo- and hyperoxic gas mixtures.

### SUMMARY OF THE INVENTION

The goal of this invention is to intensify the therapeutic and training processes, to increase the effectiveness of training using exposure to gas mixtures; to decrease the time of single treatment as well as the length of treatment or training period; to allow for customization of training settings for a given individual through varying the composition of the hypoxic mixture as well as the length of exposure depending on the condition of the patient; to improve safety and effectiveness of the device and to make the device inexpensive, straight-forward, autonomous and reliable for the process of complex interval normobaric hypo-/hyperoxic training.

The mentioned result is achieved the following way. The device for complex interval normobaric hypoxic-hyperoxic training comprising a compressor used as a source of pressure, a gas separation module, and an adjustable valve, is additionally equipped with a the gas separation module having separated outputs for hypoxic and hyperoxic mixtures. The outputs of gas separation module are connected to the input channels of the distributor, one though an ejector and another though a regulating valve.

The device may be encased together with a compressor, with a single input jet and multiple output jets corresponding to the number of simultaneous users (patients). The device may be also separated from the compressor while equipped with a respiration unit, which may be also placed at a necessary distance from the case.

It is also recommended that a moisture trap (filter) and a pressure regulator/stabilizer of incoming air pressure of the membrane apparatus be installed between the compressor and the gas separation module.

To control oxygen concentration, following the adjustable valve a tubular attachment should be installed to transport the gas mixture to an oxygen (O2) gas analyzer, wherein the output from the gas analyzer should be connected to the control unit while one of the control unit's own outputs is connected to the adjustable valve.

It is recommended to install a humidifying and/or aromatizing unit between the gas separating module and the respiration node.

It is advisable to equip this device with a distributor with one output feeding into the airflow channel connecting to the respiration node and the other venting into the atmosphere; a control unit should also be provided with unit's outputs communicating to the regulating inputs of the distributor and of the adjustable valve.

The control unit may be a microcontroller receiving signals from pulse sensor, blood hemoglobin oxygen saturation sensor, patient's attention sensor, oxygen sensor or membrane apparatus operation mode sensor; and switching the supply of hypoxic gas mixture to the hyperoxic and vice versa according to the algorithm recorded in the microcontroller's memory, also according to sensors and initial control unit menu parameters.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

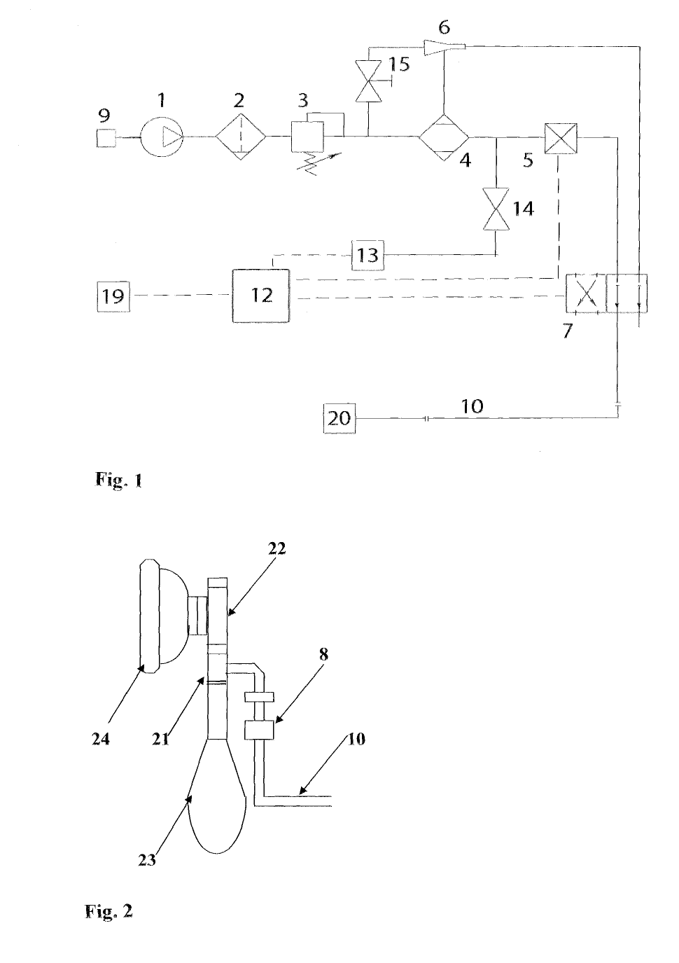

FIG. 1 depicts the proposed device in accordance with the subject invention

The device for complex interval normobaric hypoxic-hyperoxic therapy includes a compressor (1) as a source of compressed air, a moisture trap filter (2), a pressure regulator/stabilizer (3), a gas separation module (4), an adjustable valve (5) and an ejector (6). The gas separation module 4 has one input and two output channels—one for hypoxic gas mixture output and another for hyperoxic gas mixture output. The adjustable valve (5) is connected to the hypoxic output (emitting oxygen-depleted air); also connected to this output is an attachment (equipped with a throttle (14)) conveying gas mixture to the gas analyzer (13). The change in oxygen concentration in the hypoxic gas mixture ranges from 1 to 18%. Ejector (6), which is supplied from compressor (1) through the moisture trap filter (2), pressure regulator/stabilizer (3) and throttle (15)—is connected to hyperoxic output (emitting oxygen-saturated air). The change in oxygen concentration in the hyperoxic gas mixture ranges from 25 to 45%. A membrane gas separation apparatus is employed as the gas separation module. The type of membrane apparatus may vary: flat frame membrane, hollow fiber membrane, spiral membrane. An air separation device other than membrane gas separation apparatus may be also used as a gas separation module; for example, short-cycle-adsorption module, as well as other types of devices.

The air mixture passes through the valve (5) and ejector (6) into the distributor (7). One of the outputs of the distributor (7) is connected to an output jet attached to the airflow channel (10); the second distributor output vents into the atmosphere. The adjustable valve (5) and distributor (7) are connected to the outputs of a control unit (12) that regulates their operation. Three sensors of the patient's condition (27) are connected to the control unit (12): pulse sensor (27), blood hemoglobin oxygen saturation sensor (28) and patient's attention sensor (29). The gas analyzer output is also connected to the control unit (12).

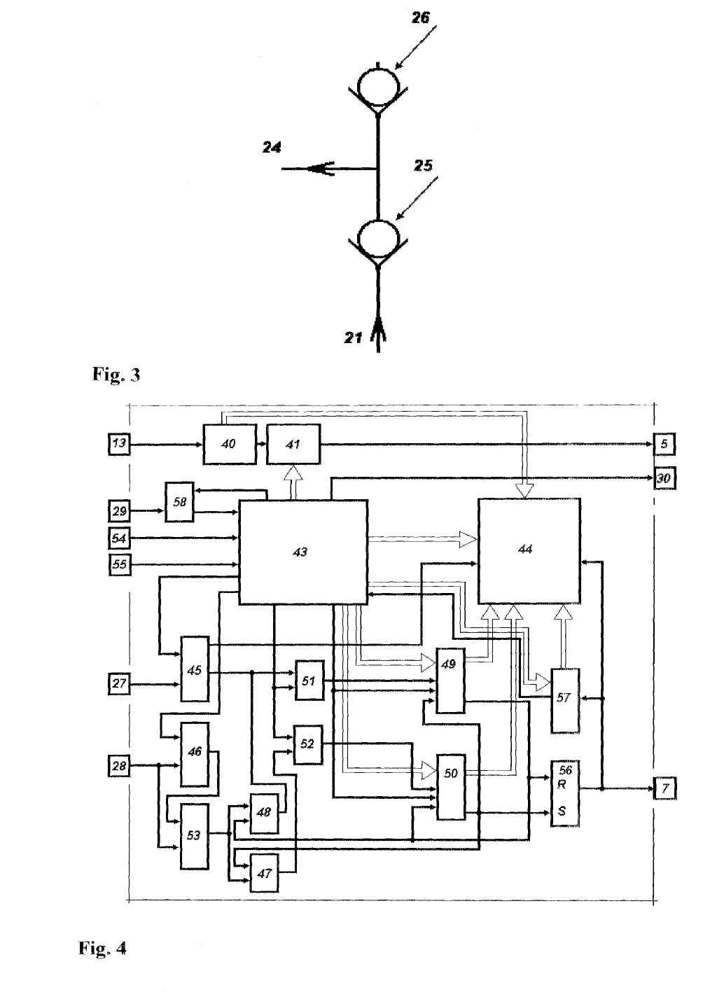

Patient's respiration node (20), depicted in FIG. 2, comprises an input jet (21), a reversible respiration valve (22) including two valves, 25 and 26, as shown in FIG. 3; a reservoir bag (23); and a respiration mask (24). A humidifier/aromatizer (8) and an airflow channel (10) are located between the distributor (7) output and the patient's respiration unit.

The device operates as follows. The compressor (1) (FIG. 1) intakes air through the jet (9) and delivers it to the moisture trap filter (2), where the air (compressed to pressure PI) is purified of mechanical debris and condensed moisture. Purified air is then supplied to the pressure stabilizer (3) and from there—to the gas separation apparatus (4) input. Due to pressure change, air is passed though the membrane in the gas separation apparatus. The air that has permeated the membrane is thus saturated with oxygen, and the remedial air (which has not permeated the membrane) is saturated with nitrogen. The nitrogen concentration of the remedial air depends upon the following:

1. The ratio of the rate of oxygen permeation to the rate of nitrogen permeation via the membrane (selectivity coefficient), which is an intrinsic characteristic of the membrane apparatus.

2. The pressure of the air supplied to the membrane apparatus.

3. The ratio of the amount of flow, which has permeated via the membrane to the amount of intake flow.

The permeating flow depends on the parameters of the membrane apparatus (membrane permeability) and pressure. We consider the value of the permeating flow to be constant since the pressure stabilizer (3) stabilizes input pressure of the air supplied to the membrane apparatus. Altering the amount of supply flow, which is a sum of permeating and remedial flows, that is, by altering the amount of remedial flow, nitrogen concentration of the remedial flow can be changed. Oxygen concentration in the hypoxic mixture is controlled by the gas analyzer (13), whose output is connected to the control unit (12).

An adjustable valve (5) is installed serially after the membrane apparatus and is used to regulate the nitrogen concentration. The valve is capable of being operated manually or by means of the control unit (12) according to the readings of the gas analyzer (13).

The pressure of the remedial flow may differ only slightly from the input pressure therefore the supply of the flow to the patient does not require effort. The pressure of the permeating flow, however, differs slightly from atmospheric to supply the flow to the patient's respiration node (20). To overcome this condition the permeating (hypoxic) flow channel is equipped with an ejector (6), which is supplied with compressed air from the pressure stabilizer. Compressed air flow creates a low pressure in the permeating flow zone thus pulling the hyperoxic flow from the membrane apparatus under pressure, sufficient to overcome compressed-air resistance of the distributor (7), humidifier/aromatizer (8), airflow channel (10) and valves (25) and (26) of the respiration node (20). Both hypoxic and hyperoxic flows are directed into the distributor (7), which is operated by the control unit (12). The distributor (7) has two input channels and two output channels. Hypoxic and hyperoxic outputs of the membrane apparatus (4) are connected to the distributor inputs: hyperoxic output is connected via the ejector (6) and hypoxic output—via the adjustable valve (5). Airflow channel (10) is connected to one of the two distributor (7) outputs and used for delivery of gas mixture to the patient's respiration unit (20); the respiration unit may be located at some distance from the device. The second distributor output channel vents into the atmosphere, so the parameters of membrane apparatus operation would not change when a switch occurs between the flows. In other words, when hypoxic mixture is supplied to the respiration unit, the hyperoxic mixture is vented into the atmosphere, and vice versa.

The patient's respiration unit (20) includes an input jet (21), which is sufficient for airflow channel connection, a reversible valve (22), a reservoir bag (23), and a respiration mask (24). A hypo- or hyperoxic flow enters the input jet (21) from the airflow channel (10) and is distributed between the reservoir bag (23) and the reversible respiration valve (22). The first valve (25) of the reversible respiration valve (22) has a slight pneumatic resistance and therefore part of the flow accumulates in the reservoir bag (23) rather than exiting though the mask. On entry, the exit valve (26) closes and the patient receives flows from the reservoir bag (23) and the airflow channel (10). On exit, the exit valve (26) opens and the valve (25) closes, and entire flow is directed into the reservoir bag (23).

The number of patient's respiration units is limited only by the efficiency of the hypo- and hyperoxic mixture generation device. The amount of gas mixture required for adequate breathing is no less than 12 liter per minute per patient. Each respiration node is connected to its own distributor (7).

Patient's condition control (27) is comprised of a pulse sensor (27), a blood hemoglobin oxygen saturation sensor (28), and patient's attention sensor (29). All sensors are connected to the control unit (12).

The control unit (12) utilizes a microcontroller and operates as follows. When the device is turned on, a menu is displayed on the control unit screen (indicator unit) that comprises the following items: setting for oxygen content of hypoxic mixture, setting for exposure time for hypoxic and hyperoxic mixtures, number of periods of exposure to the “hypoxic gas mixture-hyperoxic gas mixture” cycle, manual to automatic switch, upper and lower limits for patient's pulse, minimal (limiting) value for patient's blood hemoglobin oxygen saturation level, and maximal time of exposure upon patient's reaching the limiting values for pulse and blood hemoglobin oxygen saturation. After all required settings are entered the microcontroller program is executed.

The control unit (12) includes a menu unit (43) through which the time of patient's exposure to hyper-/hypoxic gas mixture is set. Also, oxygen content of hypoxic gas mixture, upper and lower limits for patient's pulse and patient's blood hemoglobin oxygen saturation values are set through the menu unit. All entered settings are reflected by the indicator unit (44).

Oxygen content in hypoxic gas mixture is regulated as follows. A signal from the gas analyzer (13) enters an input of an amplifier-normalizer (40). The oxygen content value is conveyed from the amplifier (40) to the indicator unit. A signal proportional to the hypoxic mixture oxygen content is transmitted from the amplifier's (40) second output to the proportional integral-differential regulator (41)—PID regulator. The PID regulator (41) controls the adjustable valve (5) according to the values sent to the PID regulator (41) from the menu unit.

Switching between hypo- and hyperoxic gas mixtures is accomplished as follows. Using menu unit, values for exposure time to either hypoxic or hyperoxic gas mixture are entered into timers (49) and (50), respectively, and the number of periods of switching from hypoxic exposure to hyperoxic exposure is entered into the counter (57).

When the manual/automatic switch is in “MANUAL” position, the output of the menu unit (43), going to logical elements “AND” (51) and (52), is low. Therefore, output of the logical elements “AND” (51) and (52) is also low, and the hypoxic mixture exposure timer (49) and hyperoxic mixture exposure timer (50) operate autonomously. At the starting time, timers (49) and (50) are off and trigger (56) is in RESET position. Thus the output signal of the trigger (trigger is controlled by the distributor (7)) is low and the hyperoxic gas mixture is delivered to the patient. When “Start” button is pressed, an impulse of generated by the menu unit (43) output, initializing operation of timers (49) and (50). The output signal of the trigger (56) is now high. The distributor (7) switches and now hypoxic gas mixture is delivered to the patient. The indicator unit (44) reflects the state of the trigger (56). After the time entered by the menu unit (43) for the timer (49) has elapsed, the timer's (49) output is high, which resets the trigger (56) to the low level state. The distributor (7) switches and hyperoxic gas mixture is delivered to the patient. High output of the timer (49) turns the timer (50) on. After the time entered by the menu unit (43) for the timer (50) has elapsed, the output generated by the timer (50) is high, which switches the trigger (56) to “ON” mode; the trigger's output is now high. The same signal turns the timer (49) on and high output signal of the trigger (56) causes the distributor (7) to switch, so hypoxic gas mixture is delivered to the patient. Each switch between low and high levels of the trigger (56) lowers the counter (57) value (which is pre-set by the menu unit (43)) by 1. When the counter (57) value reaches 0, a signal is generated at the counter output and transmitted to the menu unit (43). The menu unit (43) then returns to its starting state and signals termination of operation of the timers (49) and (50). The states of the counter (57) and the timers (49) and (50) are reflected by the indicator unit (44).

When the manual/automatic switch is in “AUTOMATIC” position, the output of the menu unit (43), going to logical elements “AND” (51) and (52), is high. This allows for control signals to be delivered to the hypoxic mixture exposure timer (49) and hyperoxic mixture exposure timer (50), wherein the timers' operation is dependant on input levels of logical elements (51) and (52). At the starting time, the timers (49) and (50) are off and the trigger (56) is in RESET position. Thus the output signal of the trigger (trigger is controlled by the distributor (7)) is low and hyperoxic gas mixture is delivered to the patient. When “Start” button is pressed an impulse of generated by the menu unit (43) output, initializing operation of the timers (49) and (50). The output signal of the trigger (56) is now high. The distributor (7) switches and hypoxic gas mixture is delivered to the patient. The indicator unit (44) reflects the state of the trigger (56). Preset values for upper and lower pulse limits and blood hemoglobin oxygen saturation are transmitted to comparison modules (45) and (46), respectively. The other inputs of comparison modules (45) and (46) receive signals from the pulse sensor (27) and the blood hemoglobin oxygen saturation sensor (28), respectively. If either upper or lower pulse limit is reached, a signal is generated at the output of the comparison module (45) and the timer (49) is turned off via the logical element “AND” (51). The trigger (56) is reset and the distributor 7 switches so hyperoxic gas mixture is delivered to the patient. The signal of the patient's pulse being outside the range of preset limits is simultaneously transmitted to the indicator unit (44).

If a signal of a value outside the range of limits preset in the menu unit (43) is received from the blood hemoglobin oxygen saturation sensor, a signal from the comparison module (46) activates the timer (53)—timer of the maximal exposure at limiting values. The time (duration) value of the timer (53) depends on the limit value entered into the comparison module (46) and the rate of change of the parameter values of the sensor (28). When the time of operation of the timer (53) elapses, a high level signal is generated at timer's output turns on the timer (49) if the timer (50) was active previously (or timer the (50) if the timer (49) was active previously). The trigger (56) switches accordingly and switches the distributor (7) to appropriate mode, so hyperoxic or hypoxic gas mixture is delivered to the patient, depending on which timer is active at the time. If no signal is received from the comparison modules while timers (49) and (50) are active, then when time of operation of the timers elapses, mode of operation of the control unit becomes analogous to “MANUAL”. Each switch between low and high signal levels of the trigger (56) lowers the counter (57) value (preset by the menu unit (43)) by 1. When the counter (57) value reaches 0, a signal is generated at the counter output and transmitted to the menu unit (43). The menu unit (43) then returns to its starting state and signals termination of operation of timers (49) and (50). The states of the counter (57) and the timers (49) and (50) are reflected by the indicator unit (44). Patient's attention sensor operates as follows: at each time period, t, menu unit transmits a signal to a light-emitting diode (LED, light signal) (30) or/and to the indicator unit (44) and to the safety timer (58). If the patient does not react to the light signal (30) or to the indicator unit (44) message, and does not close contacts of sensor (29), then after time t1 elapses, timer (58) will transmit a signal to the menu unit, terminating operation of the control unit (12), initiating delivery of hyperoxic gas mixture to the patient and displaying patient's condition signal on the indicator unit.

During the first period, the control unit directs the distributor (7) to deliver hypoxic gas mixture to the patient's respiration node while hyperoxic gas mixture is vented into the atmosphere.

If “MANUAL” mode is selected on the control unit (12), after the exposure time preset by the initial menu elapses, the control unit will direct the distributor (7) to deliver hyperoxic gas mixture to the patient's respiration node while hypoxic gas mixture is vented into the atmosphere. After the time of exposure to hyperoxic gas mixture elapses, the control unit will switch the distributor to deliver hypoxic gas mixture. The process will continue for the number of periods set in the menu, after which the control unit will signal the end of the procedure.

If “AUTOMATIC” mode is selected, after the program is initiated, the control unit responds to the signals from the pulse sensor (27), the blood hemoglobin oxygen saturation sensor (28) and the patient's attention sensor (29). During the first period, the control unit directs the distributor (7) to deliver hypoxic gas mixture to the patient's respiration node while hyperoxic mixture is vented into the atmosphere. If during the period of exposure to hypoxic gas mixture the patient's pulse registers outside the range of preset upper and lower limits, control unit will unconditionally switch the distributor (7) to deliver hyperoxic gas mixture to the patient's respiration node.

If during the period of exposure to the hypoxic gas mixture the value of blood hemoglobin oxygen saturation reaches the preset minimal acceptable limit with deviation within 5% of this value, the time of exposure is prolonged by 1 minute, and then the delivery is switched to the hyperoxic gas mixture.

If during the period of exposure to the hypoxic gas mixture the value of blood hemoglobin oxygen saturation drops below the preset limit with a deviation of more than 5%, the timer of the maximal possible exposure time at preset limit will begin counting down. This will cut down the 1-minute prolongation of the time of hypoxic mixture delivery. In case of further drop in the value of blood hemoglobin oxygen saturation, the dependence of exposure time on blood oxygen saturation will become linear so that at a 10% deviation from the preset minimal oxygen saturation value time of exposure will be 0. Upon reaching zero time of exposure, control unit will switch the distributor (7) to deliver hyperoxic gas mixture to the patient's respiration node.

When the high value of patient's blood hemoglobin oxygen saturation is reached during exposure to hyperoxic gas mixture, the control unit (12) will initiate the count down timer of maximal exposure time at preset limit. When exposure time reaches 0, the control unit (12) will switch the distributor (7) to deliver hypoxic gas mixture to the patient's respiration node. If the limiting values of either blood oxygen saturation or pulse are not reached during the period of exposure, the exposure timer will switch the distributor (7) to mode of delivery opposite to previous. The process will continue for the number of periods of exposure set in the menu.

The patient's attention sensor (29) is intended to assure patient's alertness and safeguard against patient fainting. For this purpose, the control unit transmits a signal to the signal light (LED) and if the patient does not react to the signal, the control unit (12) will switch the distributor (7) to deliver hyperoxic gas mixture to the patient's respiration unit and a danger signal will be transmitted by the control unit.

Thus this device for complex interval normobaric hypoxic-hyperoxic training intensifies therapeutic and training processes, increases effectiveness of the method and decreases time of a single procedure as well as total time requirement for treatment or training, and provides an option for individual customization of the hypoxic mixture composition while ensuring further safety of the patient undergoing training and restorative procedures.

While this invention has been particularly shown and described with references to preferred embodiments thereof, it will be understood by those skilled in the art that various changes in form and details may be made therein without departing from the scope of the invention encompassed by the appended claims.

What is claimed is:

1. A device for complex interval normobaric hypoxic-hyperoxic training comprising:
a source of compressed air;
a gas separation apparatus connected with the source of compressed air, the gas separation apparatus having a hyperoxic output and a hypoxic output, and being used as a gas separation module;
an adjustable valve connected to the hypoxic output of the gas separation module; and
a distributor for periodically delivering hyperoxic and hypoxic gas mixtures to a patient's respiration device;
wherein one input of the distributor is connected with the adjustable valve, the other input of the distributor being associated with the hyperoxic output of the gas separation module;
wherein one output of the distributor is connected with an input of a device adapted for linking to the patient's respiration device; and
wherein the other output of the distributor communicates with the ambient atmosphere.

2. The device according to claim 1, wherein the distributor is capable of being switched such that the input of the distributor previously connected with the adjustable valve is connected with the hyperoxic output of the gas separation module, and visa versa.

3. The device according to claim 1, wherein the hyperoxic output of the gas separation module is associated with the distributor via an ejector of a hyperoxic gas mixture to accomplish delivery of hyperoxic mixture to the patient's respiration device; wherein one input of the ejector is connected with the source of compressed air; wherein the other input of the ejector is connected with the hyperoxic output of the gas separation module; and wherein the output of the ejector is connected with the distributor.

4. The device according to claim 2, further being enclosed in a case, wherein the case comprises a single input jet and a plurality of output jets, corresponding to the number of simultaneously serviced users.

5. The device according to claim 1, wherein the respiration block is located outside the device and is equipped with a humidifier, a reversible valve, a reservoir bag and a face mask.

6. The device according to claim 1, further comprising a moisture trap filter installed between the source of compressed air and the gas separation module.

7. The device according to claim 1, further comprising a gas analyzer for controlling an oxygen concentration in a gas mixture produced by the gas separation module, wherein the gas analyzer is connected with an executive unit of the adjustable valve via a control unit.

8. The device according to claim 1, further comprising a unit for measuring values of a patient's pulse and blood hemoglobin oxygen saturation, wherein the unit for measuring is connected to the distributor via the control unit, and wherein these values being compared with predefined threshold levels preset in a microcontroller are used as criteria for switching from delivery of hypoxic gas mixture to hyperoxic, and vice versa.

9. The device according to claim 1, further comprising a control unit connected with the distributor and an executive unit of the adjustable valve.

10. The device according to claim 1, further comprising a unit for checking a patient's condition status, wherein the unit for checking is connected with the distributor via the control unit.

11. The device according to claim 1, wherein the gas separation module is implemented as one of the group consisting of a hollow-fiber membrane apparatus, flat frame membrane apparatus, and spiral wound membrane apparatus.

12. The device according to claim 1, wherein the gas separation module is implemented as a short-cycle-adsorption device.
